(19) United States
(12) Patent Application Publication
(10) Pub. No.: US 2002/0015685 A1
(43) Pub. Date: Feb. 7, 2002

(54) Enhanced deposition of hair and skin benefit agents from water-in-oil-in-water conditioner compositions

(75) Inventors: Fe P Pascual, Elk Grove Village, IL (US); Gerald Patrick Newell, Hoffman Estates, IL (US); Wei-Mei Sun, Palatine, IL (US); Tirucherai V Vasudevan, Lake Zurich, IL (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 09/854,400

(22) Filed: May 11, 2001

Related U.S. Application Data
(60) Provisional application No. 60/204,055, filed on May 12, 2000.

(51) Int. Cl. A61K 7/075; A61K 7/08

(52) U.S. Cl. 424/70.22; 424/70.28

(57) ABSTRACT

A rinse-off water-in-oil-in-water (W₁/O/W₂) multiple emulsion composition comprising:

1) about 5% to about 99% by weight of the total composition of an external aqueous phase comprising water; wherein said external aqueous phase further comprises a liquid crystalline conditioning agent which comprises a fatty alcohol or fatty acid or a mixture thereof; and a quaternary ammonium compound, wherein at least one of the fatty alcohol, fatty acid or quaternary ammonium compound, contains an unsaturated hydrocarbon chain;

2) about 1% to about 95% by weight of the total composition of a primary water-in oil (W₁/O) emulsion, said primary water-in-oil (W₁/O) emulsion comprising a water soluble benefit agent;

3) about 0.5% to about 95% by weight of the primary emulsion of a phase oil phase comprising a volaltile silicone or volatile hydrocarbon compound; and

4) about 0.1% to about 20% by weight of the primary emulsion of a surfactant phase comprising an oil-soluble silicone-based or silicone-free surfactant is described.

### BACKGROUND OF THE INVENTION

Achieving deposition of desirable levels of water soluble benefit agents from rinse-off hair care products has posed a problem. More specifically, when hair care products are rinsed off with water, much of the water soluble benefit agents are rinsed off as well. The present invention addresses this problem.

Patent publications which relate to the present invention include:

U.S. Pat. Nos. 5,656,280 and 5,589,177 which disclose water-in-oil-in water emulsion compositions which comprise an external aqueous phase optionally incorporating a surfactant system capable of forming liquid crystals as an emulsifier. The internal phase comprises a water-in-oil emulsion.

### SUMMARY OF THE INVENTION

The present invention relates to a rinse-off water-in-oil-in-water (W₁/O/W₂) multiple emulsion hair care composition comprising:

(1) about 5% to about 99% by weight of the total composition of an external aqueous phase comprising water; wherein said external aqueous phase further comprises a liquid crystalline conditioning agent which comprises a of fatty alcohol or a fatty acid or a mixture thereof; and a quaternary ammonium compound; wherein at least one of the fatty alcohol, fatty acid or the quaternary ammonium compound, contains an unsaturated hydrocarbon chain;

(2) about 1% to about 95% by weight of the total composition of a primary water-in-oil (W₁/O) emulsion, said primary water-in-oil (W₁/O) emulsion comprising a water soluble benefit agent;

(3) about 0.5% to about 95% by weight of the primary emulsion of an oil phase comprising a volatile silicone or volatile hydrocarbon compound; and

(4) about 0.1% to about 20% by weight of the primary emulsion of a surfactant phase comprising an oil-soluble silicone-based or silicone-free surfactant.

The invention also relates to a method of achieving a high level of water soluble benefit agent deposition on hair or on skin, which comprises contacting said hair or skin with a rinse-off water-in-oil-in-water multiple emulsion hair care composition as described just above.

### DETAILED DESCRIPTION OF THE INVENTION

As used herein “topically-active benefit agent or compound” is used interchangeably with benefit agent.

The present invention relates to a rinse-off water-in-oil-in-water (W₁/O/W₂) multiple emulsion hair care composition comprising:

(1) about 5% to about 99% by weight of the total composition of an external aqueous phase comprising water; wherein said external aqueous phase further comprises a liquid crystalline conditioning agent which comprises a fatty alcohol, a fatty acid or a mixture thereof; and a quaternary ammonium compound, wherein at least one of the fatty alcohol, fatty acid or the quaternary ammonium compound, contains an unsaturated hydrocarbon chain;

(2) about 1% to about 95% by weight of the total composition of a primary water-in-oil (W₁/O) emulsion, said primary water-in-oil (W₁/O) emulsion comprising a water soluble benefit agent;

(3) about 0.5% to about 95% by weight of the primary emulsion of an oil phase comprising a volatile silicone or volatile hydrocarbon compound; and

(4) about 0.1% to about 20% by weight of the primary emulsion of a surfactant phase comprising an oil-soluble silicone-based or silicone-free surfactant.

Preferred compositions of the invention are those wherein the quaternary ammonium compound in said liquid crystalline conditioning agent contains at least one unsaturated hydrocarbon chain attached to the quaternary nitrogen.

A topically-effective composition used in the present invention is a stable, water-in-oil-water, W₁/O/W₂ multiple emulsion comprising an external aqueous phase and an internal primary emulation. Water-in-oil-in-water emulsions are described in U.S. Pat. No. 5,589,177 which is hereby incorporated by reference. As can be seen below, U.S. Pat. No. 5,589,177 is referred to a number of times in the present application to supply details regarding the preparation of compositions of the present invention. The primary emulsion of the present invention comprises a first water-soluble, topically-active benefit agent, such as a hair dye; a surfactant phase comprising a silicone-free surfactant or surfactant blend having an HLB value of about 10 or less, an oil-soluble silicone-based surfactant, an oil-soluble polymeric surfactant, or a mixture thereof; an oil phase comprising a volatile silicone compound, a nonvolatile silicone compound, a volatile hydrocarbon compound, a nonvolatile hydrocarbon compound, or a mixture thereof; and water.

The W₁/O/W₂ multiple emulsion compositions are rinse-off compositions. The W₁/O/W₂ multiple emulsion composition can be liquid having a viscosity in the range of about 1,000 to about 50,000 cps. The multiple emulsion compositions also can be a cream having a viscosity of about 50,000 to about 1,200,000 cps. The external phase of the W₁/O/W₂ multiple emulsion composition is essentially free of a polymeric gelling agent.

After application to hair and skin, the external aqueous phase is rinsed off from the skin or hair. The oil phase of the primary emulsion, if sufficiently volatile, evaporates during the skin or hair drying process to release the internal aqueous phase of the primary emulsion, thereby allowing the first topically-active compound to contact the skin or hair and perform its intended function. Alternatively, the internal aqueous phase is released after the rinsing step by rubbing the skin or hair to break the primary emulsion.

The water-in-oil-in-water multiple emulsion compositions used in the present invention are designated herein as W₁/O/W₂ multiple emulsions. This designation indicates that an oil phase (O) separates an encapsulated, discontinuous aqueous phase (W₁) from a continuous aqueous phase (W₂). The two aqueous phases (that is W₁ and W₂) can differ in compositional make-up, but the WW₁ and W₂ phases can also be identical.

In accordance with an important feature of the present invention, the liquid W₁/O/W₂ multiple emulsion compositions are stable compositions that effectively resist droplet coalescence over long storage periods. The W₁/O/W₂ compositions also resist leakage or transfer of water-soluble components between the internal aqueous phase of the primary emulsion (W₁) and external aqueous phase (W₂). A W₁/O/W₂ multiple emulsion composition generally is available for immediate application to the skin or hair without the need to shake or agitate the composition in order to redisperse composition ingredients throughout the composition prior to use.

In particular, the W₁/O/W₂ multiple emulsion compositions comprise:

(a) about 5% to about 99% by weight of the composition of an external aqueous phase; and

(b) about 1I% to about 95%, by weight of the composition of a primary W₁/O emulsion.

The primary W₁/O emulsion comprises:

(a) about 1% to about 95% by weight of the primary emulsion (W₁/O) of an aqueous phase comprising (i) water and (ii) an effective amount of a benefit agent, typically about 0.1% to about 90% by weight of the primary emulsion (W₁/O);

(b) about 0.5% to about 95% by weight of the primary emulsion of an oil phase comprising a volatile silicone compound, a nonvolatile silicone compound, a volatile hydrocarbon compound, a nonvolatile hydrocarbon compound, or a mixture thereof; and

(c) about 0.1% to about 20% by weight of the primary emulsion of a surfactant phase comprising a silicone-free surfactant or surfactant blend having an HLB value of about 10 or less, and preferably about 7 or less, of an oil-soluble silicone-based surfactant, an oil-soluble polymeric surfactant, or a mixture thereof.

The W₁/O/W₂ multiple emulsion hair care compositions of the invention exhibit exceptional aesthetic and functional properties. The W₁/O/W₂ multiple emulsion compositions are liquids or creams. The W₁/O/W₂ multiple emulsion compositions of the invention which comprise the unsaturated quaternary ammonium compounds recited herein, provide enhanced deposition on hair of benefit agent, for example, hair dyes and colorants; and enhanced deposition on skin of skin benefit agents.

I. Primary Water-in-Oil (W₁/O) Emulsion

The primary W₁/O emulsion comprises water, a cosmetic benefit compound, an oil phase and a surfactant phase. The water and cosmetic benefit agent comprise the aqueous phase of the primary W₁/O emulsion. The cosmetic benefit agent may be a cosmetic hair colorant, a hair styling polymer, glycerin, or another hair benefit agent. The primary emulsion comprises droplets containing water and the cosmetic benefit agent (i.e., the aqueout phase). The droplets containing water and the cosmetic benefit agent (W₁) have a diameter ranging from about 1 to about 75 μm and are enveloped by a membrane or film comprising the oil phase and the surfactant phase. The contents of the internal aqueous phage (W₁) therefore do not contact the external aqueous phase (W₂) of the W₁/O/W₂ multiple emulsion.

The primary W₁/O emulsion is present in a W₁/O/W₂ multiple emulsion composition in an amount of about 1% to about 95%, and preferably about 5% to about 50% by weight of the multiple emulsion composition. To achieve the full advantage of the present invention, the primary W₁/O emulsion is present in an amount of about 10% to about 45%, by weight of the multiple emulsion composition.

A. The Aqueous Phase

The aqueous phase is the internal phase (W₁) of the primary W₁/O emulsion. The internal aqueous phase constitutes about 1% to about 95% by weight of the primary emulsion, and the relatively small amounts of oil phase and surfactant phase separate the internal aqueous phase (W₁) of the primary emulsion from the external aqueous phase (W₂).

The aqueous phase (W₁) of the present W₁/O/W₂ multiple emulsion compositions comprises water and a benefit agent, for example, a hair colorant. The aqueous phase (W₁) can further comprise additional topically-active compounds and/or optional water-soluble compounds capable of providing a desired aesthetic or functional effect, such as a fragrance.

The aqueous phase (W₁) (which is also referred to as the first phase) comprises about 1% to about 95%, and preferably about 10% to about 95%, by weight of the primary emulsion. To achieve the full advantage of the present invention, the aqueous phase comprises about 25% to about 95% by weight of the primary emulsion. Alternatively expressed, the internal aqueous phase(W₁) is present in an amount of about 0.01% to about 57%, preferably about 0.5% to about 47.5%, and most preferably about 2.5% to about 42.75% by weight of the W₁/O/W₂ multiple emulsion composition.

1 . Materials Used in the Internal Aqueous Phase (W₁)

The compounds used in the internal aqueous phase of the invention can be any water soluble hair care or skin care benefit agents such as cosmetic hair colorants. These compounds are present in a sufficient amount to perform the intended function, typically in an amount of about 0.1% to about 90% by weight of the primary emulsion, or, alternatively expressed, about 0.001% to about 17% by weight of the W₁/O/W₂ multiple emulsion composition.

Because of the structure of the multiple emulsion, the cosmetic hair colorant typically remains on the hair after application, as opposed to being rinsed off from the hair shortly after application.

In accordance with an important feature of the present invention, the water soluble hair care or skin care benefit agent can be incorporated into the aqueous phase or into the oil phase of the primary emulsion. Whether a particular cosmetic hair colorant, for example, is incorporated into the aqueous phase or the oil phase of the primary emulsion is related to the solubility of the cosmetic colorant in water. In preferred embodiments, the first cosmetic colorant is water soluble and is incorporated into the internal aqueous phase (W₁).

As used herein, the term “water soluble” means water soluble or water dispersible. A water-soluble compound has a water solubility of at least 0.1 g (grams) per 100 ml (milliliters) of water and forms a true solution. A water-soluble compound can be inherently water soluble or can be made water soluble by the addition of a solubilizing compound, such as a coupling agent, a co-surfactant or a solvent. A water-dispersible compound remains dispersed in water for at least the time period necessary to manufacture the primary W₁/O emulsion, i.e., at least about one hour.

In addition, the cosmetic hair colorant, for example, can be incorporated into the aqueous phase and another topically-active compound can be incorporated into the oil phase to achieve enhanced efficacy or to provide different benefits.

The cosmetic hair colorant therefore can be one of, or a combination of, any compound that is useful as a cosmetic colorant upon treating the hair. Such compounds include hair colorants, and the like. The first compound is preferably a certified colorant which will prove safe to all consumers. Specific preferred certified colorants include, among others, F D 8 C Blue 1; D & C Orange 4; D & C Red 33, D & C Brown 1; Ext. D & C Violet 2; F D & C Red 40; and mixtures thereof. An advantage of these cosmetic colorants is that they do not need peroxide to be activated. Other specific cosmetic colorants employed in the invention are the water soluble colorants listed in the CTFA Handbook, on pages 70-71, which is hereby incorporated by reference.

The colorant can be a water-soluble hair colorant, such as, but not limited to, water-soluble cationic dyes (which may be non-certified), water-soluble F D & C dyes, like Blue No. 1, Blue No. 2, Red No. 3, Red No. 4, or Red No. 40, water-soluble D & C dyes, like Yellow No. 10, Red No. 22 or Red No. 28; or pyrogallol or resorcinol or mixtures of any of the above.

2. Water

Sufficient water is present in the aqueous phase such that the aqueous phase comprises about 1% to about 95% by weight of the primary emulsion. Total water present in the W₁/O/W₂ multiple emulsion composition is about 5% to about 99.9% and typically about 40% to about 95% by weight of the composition.

3. Optional Ingredients

The internal aqueous phase also can include optional ingredients traditionally included in topically-applied compositions. These optional ingredients include, but are not limited to, dyes, fragrances, preservatives, antioxidants, detackifying agents, and similar types of compounds. The optional ingredients are included in the internal aqueous phase of the primary emulsion in an amount sufficient to perform their intended function.

B. The Oil Phase

The primary W₁/O emulsion also comprises about 0.5% to about 95%, preferably about 0.5% to about 80%, and most preferably about 0.5% to about 75% by weight of the primary emulsion of an oil phase; alteratively expressed as about 0.005% to about 57%, preferably about 0.005% to about 48%, and most preferably about 0.005% to about 45% by weight of the W₁/O/W₂ multiple emulsion composition. The oil phase encapsulates the internal aqueous W₁ phase of the primary emulsion to form droplets of about 5 to about 1,000 μm, and preferably about 10 to about 500 μm, in diameter. The oil phase therefore provides a barrier between the internal aqueous phase of the primary emulsion and the external aqueous phase of the W₁/O/W₂ multiple emulsion composition. The oil phase provides a sufficient barrier even though the oil phase can constitute as little as 0.5% by weight of the primary emulsion.

The oil phase can be a volatile oil phase, a nonvolatile oil phase, or a mixture thereof. A volatile oil phase comprising a volatile silicone compound, a volatile hydrocarbon compound, or a mixture thereof is preferred, and evaporates from the skin or hair after rinsing to release the first topically -active benefit agent. A nonvolatile oil phase, comprising a nonvolatile silicone compound, a nonvolatile hydrocarbon compound, or a mixture thereof, remains on the skin or hair after the rinsing step and the first topically-active compound is released by rubbing the skin or hair to break the primary W₁/O emulsion.

In accordance with an important feature of the present invention, the oil phase comprises a combination of a volatile oil phase and a nonvolatile oil phase. In this embodiment, an oil phase can be designed to evaporate at a pre-selected temperature and provide a controlled release of the first topically-active compound at the pre-selected temperature. Pre-selected temperatures are those encountered during normal hair drying, provided by a hair dryer, or provided by a curling iron.

In one embodiment, the oil phase is sufficiently volatile to evaporate during the process of drying the skin or hair, and thereby release the internal aqueous (W₁) phase, which includes the first topically-active compound, to contact the skin or hair. The volatile oil phase evaporates after the external aqueous phase of the W₁/O/W₂ multiple emulsion composition is rinsed from the skin or hair.

The oil phase can comprise a volatile oil phase, a nonvolatile oil phase or a mixture thereof. The volatile oil phase comprises a volatile silicone compound, a volatile hydrocarbon compound or a mixture thereof. Preferably, the volatile oil phase comprises a volatile silicone compound. The volatile oil phase evaporates from the skin or hair to release the first topically-active benefit agent. Exemplary volatile compounds are listed in U.S. Pat. No. 5,589,177.

The cyclic, low molecular weight, volatile polydimethylsiloxanes, designated in the CTFA International Cosmetic Ingredient Dictionary, 4th Ed., Cosmetic, Toiletry and Fragrance Association, Washington, D.C. (1991) (hereinafter CTFA Dictionary) as cyclomethicones, are the preferred ailoxanea used in a composition of the present invention and are listed in U.S. Pat. No. 5,589,177.

The volatile oil phase also can comprise a volatile hydrocarbon compound. Volatile hydrocarbon compounds are listed in U.S. Pat. No. 5,589,177. The volatile hydrocarbon compounds perform the same function and provide essentially the same benefits as the volatile silicone compounds.

As previously stated, the oil phase also can be a nonvolatile oil phase. The nonvolatile oil phase comprises a nonvolatile silicone compound, a nonvolatile hydrocarbon, or a mixture thereof. Preferably, the nonvolatile oil phase comprises a nonvolatile silicone compound. The nonvolatile oil phase does not evaporate from the skin or hair. The first topically-active benefit agent therefore is released by rubbing the skin or hair to rupture the primary W₁/O) emulsion. The nonvolatile oil phase boils at atmospheric pressure at a temperature above about 250° C. Exemplary nonvolatile silicone compounds are listed in U.S. Pat. No.5,589,177.

The nonvolatile oil phase also can comprise a nonvolatile hydrocarbon compound, such as mineral oil. Other exemplary. nonvolatile hydrocarbon compounds that can be incorporated into the oil phase include, but are not limited to, a branched 1-decene oligomer, like 1-decene dimmer or a polydecene. The oil phase also optionally can comprise (1) an oil, such as jojoba oil, wheat germ oil or purcellin oil; or (2) a water-insoluble emollient, such as, for example, an ester having at least about 10 carbon atoms, and preferably about 10 to about 32 carbon atoms. Suitable esters are listed in U.S. Pat. No. 5,589,177. The surfactant phase and the external aqueous phases of compositions of the invention are described in U.S. Pat. No. 5,589,177.

2. The Secondary or External aqueous phase (W₂)

External Aqueous Phase

A surfactant system capable of forming a liquid crystal structure is used as the emulsifier in the external aqueous phase (W₂). The surfactant system can be a single surfactant, or a blend of surfactants. In some cases, a particular surfactant cannot form a liquid crystal structure alone, but can participate in the formation of liquid crystals in the presence of a second surfactant. Such a surfactant system forms a layer of lamellar liquid crystals around the primary W₁/O emulsion to provide a barrier between the primary emulsion and the external aqueous W₂ phase. This type of an emulsion is different from conventional emulsions which rely upon the orientation of the hydrophobic and hydrophilic components of a surfactant at an oil-water interface. The formation of a layer of lamellar liquid crystals around the W₁/O primary emulsion can be detected by the presence of Maltese crosses viewed by optical microscopy through crossed polarizing plates or by freeze fracture electron microscopy. As previously described, the HLB value of a surfactant is an approximate measurement of the solubility of a surfactant in water or oil. However, an HLD value is used only when the concentration of a surfactant in solution is sufficiently low such that a molecular monolayer of the surfactant is formed around droplets dispersed in a continuous phase, i.e., around the primary W₁/O emulsion. At higher surfactant concentrations, the surfactant can undergo a phase transition and the oil droplets then can be surrounded by a layer of lamellar liquid crystals, which can improve emulsion stability. The liquid crystals are localized at the oil/water interface and have been observed by microscopy techniques.

Surfactants capable of participating in the formation of a liquid crystal structure around the primary W₁/O emulsion to stabilize the W₁/O/W₂ multiple emulsion composition are listed in U.S. Pat. No. 5,589,177 which has been incorporated by reference. The surfactant systems are listed in U.S. Pat. No. 5,589,177. The external aqueous phase constitutes from about 0.1 to 30 wt. %, preferably 1 to 20 wt % and more preferably 2 to 10 wt. % surfactants.

An unsaturated hair conditioning surfactant can be incorporated into this lamellar gel phase. An unsaturated hair conditioning surfactant is a quaternary ammonium compound in which at least one hydrocarbon chain that is attached to nitrogen is unsaturated, or a fatty acid or fatty alcohol in which the alkyl chain is unsaturated. Suitable quaternary ammonium compounds or fatty acids or fatty alcohols containing an unsaturated hydrocarbon chain, are those wherein said hydrocarbon chain has 8 to 22 carbon atoms. Nonlimiting examples of such unsaturated quaternary ammonium compounds include, most preferably, ditallow dimethyl ammonium chloride, oleoyl trimethyl ammonium chloride and polyethylene glycol -2 (PEG-2) and oleammonium chloride. Preferred unsaturated fatty acids or fatty alcohol include oleic acid and oleoyl alcohol.

The unsaturated quaternary ammonium surtactant in the external aqueous phase causes increased deposition on the hair or skin of the hair or skin benefit agent in the internal aqueous phase. For this reason compositions of the invention can comprise these unsaturated quaternary ammonium surfactants in the lamellar liquid crystalline structure in the external phase. The unsaturated quaternary ammonium surfactant if present, can comprise from about 1 to 99 wt. %, preferably 5 to 90 wt. % more preferably 10 to 50 wt. % of the total surfactants present in the external aqueous phase. The unsaturated fatty acid or fatty alcohol surfactant comprises from about 0 to 99 wt. %, preferably 0 to 90 wt. % more preferably 0 to 50 wt % of the total surfactants present in the external aqueous phase

The external aqueous phase can include optional ingredients traditionally included in topically applied compositions, such as fragrances, antioxidants, detackifying agents, preservatives and dyes. Colorants included in this layer of compositions of the invention are only for supplying color to the compositions and not for dying the hair.

The W₁/O/W₂ multiple emulsion compositions of the present invention can be prepared as described in U.S. Pat. No. 5,589,177.

The primary emulsion in the compositions of the invention can be made up of the following ingredients in the following ranges set forth below. These ingredients are illustrative, and other ingredients can be included in compositions of the invention. These other ingredients are set forth through out the present specification, for example, in the above descriptions of the water and oil phases of the compositions of the invention.

| Ingredient | Weight % |
|---|---|
| Water | 50-90 |
| Cetyl Dimethicone Copolyol | 3-10 |
| Cyclomethicone | 5-25 |
| Certified Dye such as: | 0-5 |
| FD&C Green #3 | |
| FD&D Red #40 | |
| FD&C Yellow #5 | |
| D&C Brown #1 | |
| D&C Orange #4 | |
| D&C Red #27 | |
| D&C Yellow #8 | |
| D&C Red #33 | |
| FD&C Blue #1 | |
| FD&C Red #4 | |
| FD&C Yellow #6 | |
| D&C Yellow #10 | |
| D&C Yellow #8 | |

A primary emulsion to be used in compositions of the invention was made as follows:

| Ingredient | Weight % |
|---|---|
| Water | 50-90 |
| Cetyl Dimethicone Copolyol | 3-10 |
| Silicone Blend | 5-20 |
| Certified Dyes | 0.5-5 |

1) Certified dyes were weighed into an appropriate container.

2) Water was added in an amount sufficient to make 80% of the total primary emulsion.

3) The mixture was mixed until the dyes were completely dissolved (aqueous phase).

4) Cetyl dimethicone copolyol and silicone blend were weighed in a mixing container.

5) The solution was mixed with moderate agitation. This constitutes the oil phase.

6) At a very slow rate the dissolved dyes were added into the oil phase in a small amount at a time.

7) Good agitation was kept all of the time and it was made sure that the mixture was homogeneous before adding more of the aqueous phase into the batch.

8) The primary emulsion got thicker as more water was added to the oil phase and the speed of mixing was adjusted as necessary.

As was noted earlier, the external aqueous phase of compositions of the invention can comprise an unsaturated quaternary ammonium surfactant. The unsaturated quaternary ammonium surfactant brings about a multi-fold increase in deposition of hair or skin care benefit agents from the multiple emulsion rinse-off hair care compositions of the present invention.

To demonstrate the increase in deposition brought about by the presence of an unsaturated quaternary ammonium surfactant in the lamellar liquid crystalline phase of the external aqueous phase of the compositions of the invention, the six conditioner compositions below, were prepared.

Effect of Different Conditioner Bases on Color Delivery

| Conditioner Base | 1 | 2 | 3 | 4 |
|---|---|---|---|---|
| | w/w | w/w | w/w | w/w |
| Ingredient | (%) | (%) | (%) | (%) |
| Stearamidopropyl Amine | 0.50 | — | 0.75 | — |
| Cetrimonium Chloride | — | 0.84 | — | — |
| Ditallow Diammonium Chloride | — | 0.37 | — | — |
| Dicecetyl Diammonium Chloride | 1.40 | — | — | — |
| PEG-2 Oleammonium Chloride | — | — | 1.34 | — |
| Behentrimonium Chloride | — | — | — | 1.28 |
| Behentrimonium Methosulfate | — | — | 0.25 | — |
| Ethoxylated (20) Cetyl Alcohol | 0.30 | — | — | — |
| Cetyl Alcohol | 3.25 | — | 3.0 | — |
| Stearyl Alcohol | 0.70 | — | 2.0 | — |
| Cetearyl Alcohol | — | 4.0 | 0.75 | 4.0 |
| Potassium chloride | 0.10 | 0.10 | — | 0.10 |
| Disodium EDTA | 0.10 | 0.10 | 0.10 | 0.10 |
| Water | To 100 | To 100 | To 100 | To 100 |
| Color Intensity Difference | 21 | 24 | 38 | 14.4 |
| (delta E*) - W₁/O/W₂ | | | | |
| emulsion conditioner | | | | |
| Color Intensity Difference | 10 | 11 | 15 | 15.0 |
| (delta E) - non- | | | | |
| W₁/O/W₂ | | | | |
| emulsion conditioner | | | | |

*- delta E represents difference in color intensity between the untreated Yak hair and the color conditioner treated Yak hair, measured on a standard LAB scale.

These above examples show that W₁/O/W₂ from Conditioner Base #3 performs best in the above table. The only active ingredients that are present in Conditioner Base #3 and not in other conditioner bases are PEG-2 Oleammonium chloride and Behentdmonium methosulfate. The great increase in color intensity difference, which was brought about by an increase in color deposition was, therefore, caused by the presence of the quaternary ammonium surfactant PEG-2 oleammonium chloride or Behentrimonium methosulfate or both.

Effect of Different Cationic Surfactants on Color Delivery

| Conditioner Base | 3 | 5 | 6 |
|---|---|---|---|
| | w/w | w/w | w/w |
| Ingredient | (%) | (%) | (%) |
| Stearamidopropyl Amine | 0.75 | 0.75 | 0.75 |
| Cetrimonium Chloride | — | — | — |
| Ditallow Diammonium Chloride | — | — | — |
| Dicecetyl Diammonium Chloride | — | — | — |
| PEG-2 Oleammonium Chloride | 1.34 | — | 1.34 |
| Behentrimonium Chloride | 0.7 | 0.7 | — |
| Ethoxylated (20) Cetyl Alcohol | — | — | — |
| Cetyl Alcohol | 0.30 | 3.0 | 3.0 |
| Stearyl Alcohol | 2.0 | 2.0 | 2.0 |
| Cetearyl Alcohol | 0.3 | 0.3 | 0.75 |
| Potassium chloride | — | — | — |
| Disodium EDTA | 0.1 | 0.1 | 0.1 |
| Water | q.s | q.s | q.s |
| Color Intensity Difference | 38 | 15.2 | 39 |
| (delta E) - WOW | | | |

This example just above shows that of the two active ingredients, PEG-2 oleammonium chloride and behentrimonium methosulfate, it is PEC-2 oleammonium chloride (an unsaturated alkyl quaternary ammonium salt) that enhances the performance of W₁/O/W₂ conditioners I conditioners 3 and 6).

The following is a description of how compositions of the present invention may be made. There is first described how the water-in-oil primary emulsion is made

EXAMPLE (7) Primary Emulsion (Brown)

| Ingredient | w/w (%) |
|---|---|
| Cyclomethicone & dimethicone copolyol | 3.0 |
| Cetyl dimethicone copolyol | 6.0 |
| Cyclomethicone | 15 |
| Water, soft | 71 |
| Ariamor Mahogany | 2.20 |
| Ariamor Steel Blue | 1.15 |
| Ariamor Straw Yellow | 1.65 |

Primary Emulsion

To prepare the aqueous phase of the primary emulsion, the following steps were taken:

1. Water was added to a container.

2. The water was heated to about 120° F.

3. All of the dyes, in powder form were added into the container, and mixed until all of the dyes were completely dissolved.

4. The mixture was cooled to about 90° F.,

To prepare the oil phase of the primary emulsion, the following steps were taken:

1. The surfactants cyclomethicone and dimethicone copolyol; and cetyl dimethicone, were added to a different container.

2. The volatile silicone, cyclomethicone was added to this container.

3. The resulting oil phase was mixed for 10 minutes.

To prepare the primary water-in-oil emulsion the above water phase was added to the above oil phase at about 390 rpm filling rate of a pump. When emulsification was complete, the primary emulsion thus formed was passed into an IKA homogenizer and sheared at 19,000 rpm, to give the final primary emulsion.

External Aqueous Phase

To prepare the external aqueous phase the following steps were taken:

1. Water was added to a container and heated to about 180° F. to about 185° F.

2. Citric acid was added to the water with moderate agitation.

3. Stearamidopropyl dimethamine was dissolved completely in the mixture.

4. PEG-2 Oleammonium chloride & PG was dissolved in the container.

5. When the temperature was at about 165° F. to about 170° F. or above, behenyl trimonium methosulfate (behenyl TMAMS) and cetearyl alcohol were added, and the mixture was mixed at about 180° F. to about 185° F. for a half hour or more until all particles were completely dissolved.

6. More water was added without aeration.

7. Disodium EDTA in water was added.

8. Kathon was added.

9. Silicone fluid was added. Fragrance was added and pH was adjusted if necessary.

To form the final compositions of the invention, the primary emulsion was added to the external aqueous phase with use of a homogenizer to make the final product smooth.

The following two compositions of the invention, which are given for illustrative and not limiting purposes, were made.

EXAMPLE 8

| Item # | Ingredient | Actual Wt. % |
|---|---|---|
| 1 | Water, Soft | 45.000 |
| 2 | Liquid Citric Acid 50% | 0.245 |
| 3 | Stearamidopropyl Dimethylamine | 0.750 |
| 4 | 67 wt. % PEG-2 Oleamonium Chloride | 2.000 |
| | & 33 wt. % Propylene Glycol | |
| 5 | 25 wt. % Behenyl TMAMS and 75 wt. % | 1.000 |
| | Cetearyl Alcohol | |
| 6 | Cetyl Alcohol | 2.700 |
| 7 | Stearyl Alcohol | 1.800 |
| 8 | Disodium EDTA | 0.100 |
| 9 | Water, Soft | 24.325 |
| 10 | Kathon CG | 0.080 |
| 11 | DMDM Hydantoin | 0.100 |
| 12 | Potassium Hydroxide, Liquid 50% | 0.000 |
| 13 | Dimethiconol (and) Triethanolamine | 1.500 |
| | Dodecylbenzenesulfate | |
| 14 | Fragrance | 0.400 |
| 15 | Primary Emulsion | 20.000 |

EXAMPLE 12

| Item # | Ingredient | Actual Wt. % |
|---|---|---|
| 1 | Water, Soft | 44.500 |
| 2 | Liquid Citric Acid 50% | 0.245 |
| 3 | Stearamidopropyl Dimethylamine | 0.750 |
| 4 | 67 wt. % PEG-2 Oleamonium Chloride | 2.000 |
| | & 33 wt. % Propylene Glycol | |
| 5 | 25 wt % Behenyl MAMS and 75 wt. % | 1.000 |
| | Ceteraryl Alcohol | |
| 6 | Cetyl Alcohol | 3.000 |
| 7 | Stearyl Alcohol | 2.000 |
| 8 | Disodium EDTA | 0.100 |
| 9 | Water, Soft | 3.800 |
| 10 | Water, Soft, 100% Active | 20.000 |
| 11 | Kathon CG | 0.0800 |
| 12 | DMDM Hydantoin | 0.100 |
| 13 | Potassium Hydroxide, Liquid 50% | 0.025 |
| 14 | Dimethiconol (and) Triethanolamine | 1.500 |
| | Dodecylbenzenesulfate | |
| 15 | Cyclopentasiloxan, 99% Active | 2.000 |
| 16 | Fragrance | 0.400 |
| 17 | Arianor Mahogany | 0.440 |
| 18 | Arianor Steel Blue | 0.230 |
| 19 | Arianor Straw Yellow | 0.330 |
| 20 | Water, Soft, 100% Active | 17.500 |

It should be understood that the foregoing detailed description is given merely by way of illustration. Obviously, many modifications and variations of the invention as hereinbefore set forth can be made without departing form the spirit and scope thereof and therefore only such limitations should be imposed as are indicated by the appended claims.

What is claimed is:

1. A rinse-off water-in-oil-in-water (W₁/O/W₂) multiple emulsion composition comprising:
(1) about 5% to about 99% by weight of the total composition of an external aqueous phase comprising water; wherein said external aqueous phase further comprises a liquid crystalline conditioning agent which comprises a fatty alcohol, a fatty acid or a mixture thereof, and a quaternary ammonium compound; wherein at least one of the fatty alcohol, the fatty acid or the quaternary ammonium compound, contains an unsaturated hydrocarbon chain;
(2) about 1% to about 95% by weight of the total composition of a primary water-in oil (W₁/O) emulsion, said primary water-in-oil (W₁/O) emulsion comprising a water soluble skin care or hair care benefit agent;
(3) about 0.5% to about 95% by weight of the primary emulsion of a phase oil phase comprising a volatile silicone or volatile hydrocarbon compound; and
(4) about 0.1% to about 20% by weight of the primary emulsion of a surfactant phase comprising an oil-soluble silicone-based or silicone-free surfactant.

2. A composition according to claim 1 wherein said quaternary ammonium compound in said liquid crystalline conditioning agent contains at least one unsaturated hydrocarbon chain attached to the quaternary nitrogen.

3. A composition according to claim 1 wherein said water soluble benefit agent is selected from the group consisting of a skin benefit agent and a hair benefit agent.

4. A composition according to claim 3 wherein said hair benefit agent is selected from the group consisting of a hair styling polymer and a hair colorant.

5. A composition according to claim 4 wherein said hair colorant is selected from the group consisting F D & C Blue 1; D & C Orange 4; D & C Red 33, D & C Brown 1: D & C Violet 2: F D & C Red 40: Blue No. 1, Blue No. 2, Red No. 3, Red No. 4, Red No. 40, D & C Yellow No. 10, Red No. 22, Red No. 28; pyrogallol resorcinol Arianor Mahogany, Arianor Steel Blue Arianor Straw Yellow, and mixtures thereof.

6. A composition according to claim 1 wherein said fatty alcohol is selected from the around consisting of cetyl alcohol, stearyl alcohol, catearyl alcohol and mixtures thereof.

7. A composition according to claim 1 wherein said fatty alcohol is selected from the group consisting of oleoyl alcohol and fatty alcohols based on tallow or a vegetable source containing a hydrocarbon chain of from about 8 to about 22 hydrocarbons.

8. A composition according to claim 1 wherein said fatty alcohol is selected from the group consisting of oleic acid and fatty acids based on tallow or a vegetable source containing a hydrocarbon chain of from about 8 to about 22 hydrocarbons.

9. A composition according to claim 1 wherein said quaternary ammonium compound contains an unsaturated hydrocarbon chain of about 8 to about 22 carbon atoms.

10. A composition according to claim 9, wherein said unsaturated quaternary ammonium compound is selected from the group consisting of ditallow dimethyl ammonium chloride, oleoyl trimethyl ammonium chloride and PEG-2 oleammonium chloride.

11. A composition according to claim 10, wherein said quaternary ammonium compound is PEG2 Oleal ammonium chloride.

12. A method for treating hair or skin which comprises contacting said hair or skin with a composition in accordance with claim 1.
